(19) United States
(12) Patent Application Publication
(10) Pub. No.: US 2006/0235520 A1
(43) Pub. Date: Oct. 19, 2006

(54) Spinal implant apparatus, method and system

(76) Inventor: Yashdip S. Pannu, Mequon, WI (US)

Correspondence Address: Maier & Maier, PLLC, 2nd Floor, 128 North Pitt Street, Alexandria, VA, 22314, US

(21) Appl. No.: 11/405,633

(22) Filed: Apr. 18, 2006

Related U.S. Application Data
(60) Provisional application No. 60/672,544, filed on Apr. 19, 2005.

(51) Int. Cl. A61F 2/44; A61B 17/88

(52) U.S. Cl. 623/17.11; 606/90

(57) ABSTRACT

An apparatus, method and system for the treatment and management of spinal defects and inserting a spinal implant into an implantation space is described. The system may include a slotted implant, a distractor and an inserter and be employed in posterior lumbar or thoracic surgery.

This application claims the priority benefit under 35 U.S.C. 119 of U.S. provisional application No. 60/672,544 filed Apr. 19, 2005 the disclosure of which is hereby incorporated by reference in its entirety.

### FIELD OF THE INVENTION

The present invention relates to apparatus, method and system for the treatment and management of spinal defects.

### BACKGROUND OF THE INVENTION

The treatment and management of spinal defects remains one of the most challenging aspects of medicine. Spinal defects occur in a wide variety of clinical situations. They may result from spondylosis, tumor or even trauma. Preserving natural spinal motion and dynamics remains a considerable challenge. Maintaining or improving spinal sagittal balance should in theory allow the spine to be repaired at the level of correction and prevent adjacent level disease. Therefore, preserving spinal anatomy and stability are even more difficult in any surgical procedure in which a portion or complete section of disc space, vertebrae or several vertebrae is removed.

Restoring natural anatomical length and shape of any bone with spondylosis is problematic. Additionally, whenever a vertebra, part of a vertebra or disc space has to be removed, it is necessary to insert a vertebral spacer to restore the natural length and curvature of the spine as well as to either maintain or restore natural spinal dynamics or promote bony fusion. A posterior artificial disc that allows for restoration of sagittal balance is one method of restoring natural spinal dynamics. Restoring spinal anatomy and stability and promoting spinal dynamics or bone fusion are even more difficult in any surgical procedure in which a portion or complete section of a vertebrae one or portion or complete section of a vertebral body or disc space.

Often times inserts are impacted posteriorly into the implantation space with force enough to fracture the adjacent vertebral bodies or cause the implant to be placed partially into the vertebral body. The limiting factor often times is the posterior height of the disc space and wedging an insert through this part of the disc space may create longitudinal grooves within the vertebral bodies to allow for migration of the spinal insert back towards the neurologic tissue with subsequent neurologic injury or cause the surgeon to undersize the implant since he or she is gauging the posterior height of the disc space and not allowing the implant to directly interface with the cortical bony surface thereby causing a pseudoarthorosis or bony non union.

Thus, there is a need for an improved implantation system for use in posterior lumbar surgery to create an interbody implantation space while achieving or maintaining natural spinal lordosis and providing for natural spinal dynamics or bony fusion while facilitating insertion and removal.

### SUMMARY OF THE INVENTION

An apparatus, method and system for the treatment and management of spinal defects are described. A surface of an apparatus has at least one slot to receive a surgical instrument such as a distractor to facilitate implantation of the apparatus in a spine. In at least one exemplary embodiment the device may be inserted into the posterior of the spine using a distractor and an insertion device.

### BRIEF DESCRIPTION OF THE DRAWINGS

Advantages of embodiments the present invention will be apparent from the following detailed description of the preferred embodiments thereof, which description should be considered in conjunction with the accompanying drawings in which:

FIG. 1 is a rear perspective view of a lumbar segment of a spine with the dural sac retracted to the left showing a partial discectomy and the guard with the blades of the distractor and distal end of the inserter holding an implant approaching the disc space between the adjacent vertebral bodies;

FIG. 2a illustrates a top down view of an exemplary embodiment of an insert;

FIG. 2b illustrates a top down view of an exemplary embodiment of an insert;

FIG. 2c illustrates a top down view of an exemplary embodiment of an insert;

FIG. 3a illustrates a rotated side view of an exemplary embodiment of an insert;

FIG. 3b illustrates a rotated side view of an exemplary embodiment of an insert;

FIG. 3c illustrates a rotated side view of an exemplary embodiment of an insert

FIG. 4a illustrates a rear view of an exemplary embodiment of an insert;

FIG. 4b illustrates a rear view of an exemplary embodiment of an insert;

FIG. 4c illustrates a rear view of an exemplary embodiment of an insert;

FIG. 5a illustrates a top down view of an exemplary embodiment of an insert;

FIG. 5b illustrates a top down view of an exemplary embodiment of an insert;

FIG. 5c illustrates a top down view of an exemplary embodiment of an insert;

FIG. 6a illustrates a rotated side view of an exemplary embodiment of an insert;

FIG. 6b illustrates a rotated side view of an exemplary embodiment of an insert;

FIG. 6c illustrates a rotated side view of an exemplary embodiment of an insert;

FIG. 7a illustrates a rear side view of an exemplary embodiment of an insert;

FIG. 7b illustrates a rear side view of an exemplary embodiment of an insert;

FIG. 7c illustrates a rear side view of an exemplary embodiment of an insert;

FIG. 8 illustrates a rotated side view of an exemplary embodiment of an insert;

FIG. 9 illustrates a rear view of an exemplary embodiment of an insert;

FIG. 10 illustrates a rotated side view of an exemplary embodiment of an insert;

FIG. 11 illustrates an enlarged fragmentary rotated side view of an exemplary embodiment of the knurls or teeth on an insert;

FIG. 12 illustrates a rotated side view of an exemplary embodiment of an insert;

FIG. 13 illustrates a rear view of an exemplary embodiment of an insert;

FIG. 14 illustrates a rotated side view of an exemplary embodiment of an insert;

FIG. 15 illustrates a perspective view of an exemplary embodiment of the knurls or teeth of the insert;

FIG. 16 illustrates a rotated side view of an exemplary embodiment of an insert;

FIG. 17 illustrates a rotated side view of an exemplary embodiment of an insert;

FIG. 18 illustrates an enlarged fragmentary rotated side view of an exemplary embodiment of the knurls or teeth on an insert;

FIG. 19 illustrates a rear view of an exemplary embodiment of an insert;

FIG. 20 illustrates a top down view of an exemplary embodiment of an insert;

FIG. 21 illustrates a partial cross-sectional view showing an exemplary embodiment of an implant, inserter and distractor inserting an implant into the disc space;

FIG. 22 illustrates a partial cross-sectional view showing an exemplary embodiment of an implant, inserter and distractor inserting an implant across the disc space;

FIG. 23 illustrates a side view of an exemplary embodiment of an implant, a partial side view of an inserter and partial side view of a distractor;

FIG. 24 illustrates a side view of an exemplary embodiment of a distractor;

FIG. 25 illustrates a side view of an exemplary embodiment of a portion of the distractor;

FIG. 26 illustrates a side view of an exemplary embodiment of a handle of the distractor;

FIG. 27a illustrates a partial cross-sectional view showing an exemplary embodiment of a distractor spreading a disc space apart;

FIG. 27b illustrates a partial cross-sectional view showing a disc space;

FIG. 28 is a partial cross-sectional view of an exemplary embodiment of a distractor spreading apart a disc space and sliding an insert along the slots into a disc space;

FIG. 29 is a partial cross-sectional view of an exemplary embodiment of a distractor sliding away from an implanted insert into a disc space;

FIG. 30 is a partial cross-sectional view of a disc space with an exemplary embodiment of a tapered insert in a disc space;

FIG. 31 is a partial cross-sectional view of a disc space with an exemplary embodiment of an arched insert in a disc space.

### DETAILED DESCRIPTION

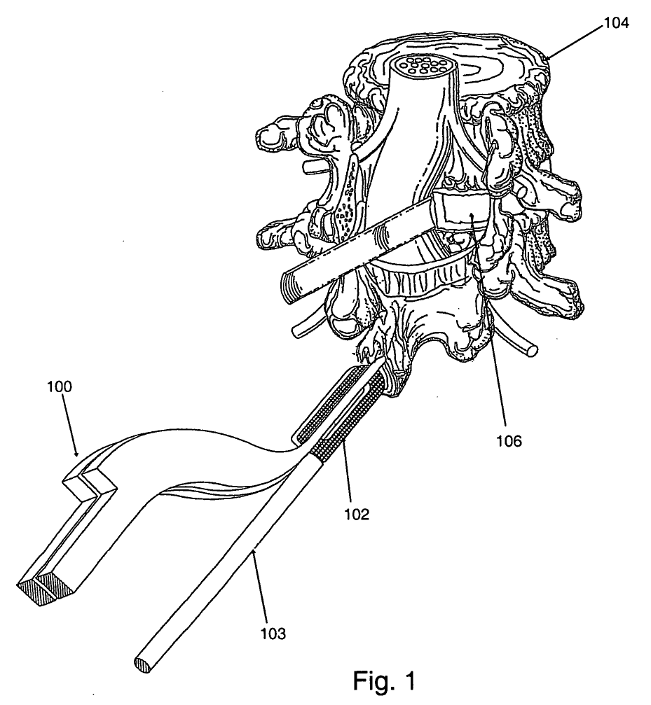

In an exemplary embodiment shown in FIG. 1, a system and method for spinal inserts is shown. In this figure a posterior view is shown with a dural sac retracted to the left, showing that a partial discectomy has been performed. In this exemplary embodiment, distractor 100 may fit around spinal insert 102 and, when inserted into a spinal column, act to separate two vertebrae by opening in a parallel fashion, allowing for the insertion of spinal insert 102. Rod or inserter 103 may be inserted into spinal insert 102. Insert 102 may have, for example, a threaded hole that accepts threading disposed on a distal end of rod 103. Rod 103 may act to hold insert 102 in place prior to its insertion in a spinal column and may also act to help position and orient insert 102 during its placing. Distractor 100 may then be positioned such that it is in line with disc space 106 in spinal column 104. Disc space 106 may be a hole formed between two vertebrae of a in a human spinal cord or thecal sac. In a further embodiment, distractor 100 may be used to implant insert 102 into hole 106 by opening the disc space in a parallel fashion. Distractor 100 may then release insert 102 and retract from hole 106 of spinal column 104 without disturbing the location, positioning or orientation of insert 102.

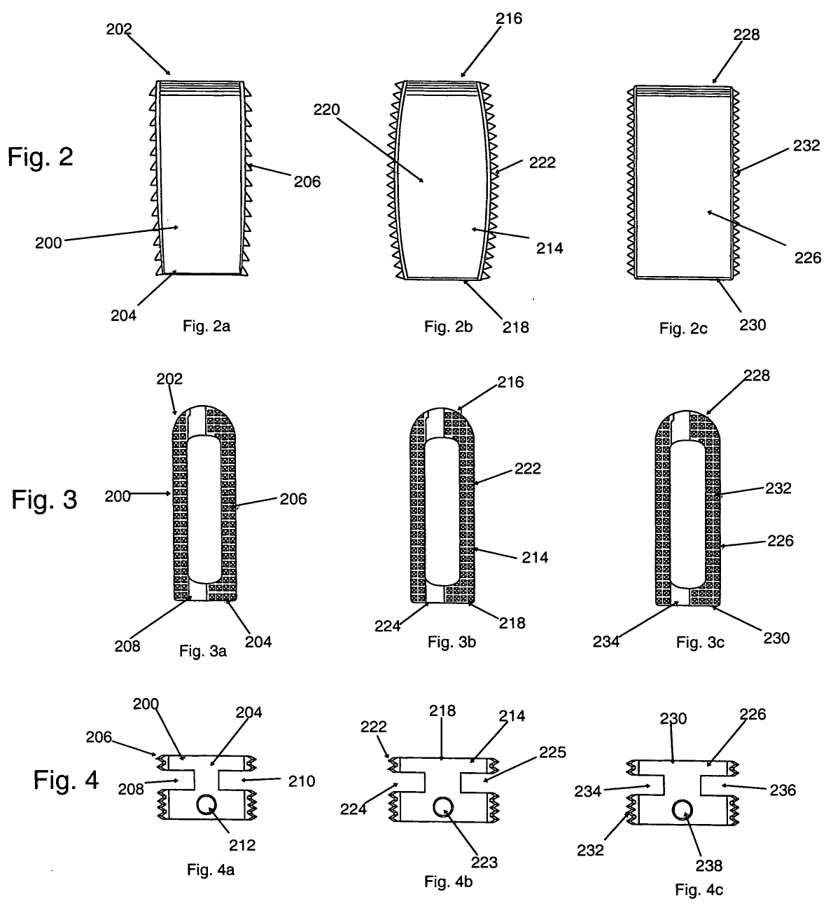

In another exemplary embodiment shown in FIG. 2, a spinal insert is shown. The spinal insert may be of any shape, for example configured in such a manner as to restore the natural height of the disc space which may have been lost due to a degenerative process. A top-down view of one side of exemplary inserts is shown in FIGS. 2a-c. For example, in FIG. 2a, insert 200 may be tapered. Here, rounded top leading portion 202 may have a width wider than bottom portion 204. Additionally, teeth or knurls 206 may be disposed on each side of insert 200. Teeth 206 may be coupled with a non-arcuate contour of a spinal insert, thus helping prevent migration or dislodging of the spinal insert. In one exemplary embodiment, teeth 206 may be angled towards bottom portion 204. In other embodiments, teeth or knurls 206 may be angled towards rounded top portion 202 or may project straight out of insert 200. Teeth 206 may act to prevent movement of insert 200 once insert 200 is inserted into, for example, the spine of a person. Additionally, insert 200, as well as any other inserts described herein, may be made out of any of a variety of materials, for example titanium, PEEK, ceramics or bone.

In FIG. 2b, another exemplary insert is shown. Insert 214 may have a rounded top portion 216 that is substantially the same width as bottom portion 218. Middle portion 220, however, may be wider than top portion 216 or bottom portion 218, giving insert 214 an arched structure. Insert 214 may also have teeth 222 disposed on each side of insert 214. Teeth or knurls 222 may project directly out of insert 214 or may be angled towards top portion 216 or bottom portion 218.

In FIG. 2c, an exemplary straight insert 218 is shown. In this embodiment, each side of insert 218 may be substantially straight. Insert 218 may also have rounded top portion 220 and substantially straight bottom portion 222. Additionally, teeth 224 may be disposed on either side of insert 218. Teeth or knurls 224 may project substantially straight out of insert 218, or may be angled towards either rounded top portion 220 or bottom portion 222.

FIG. 3 shows a further exemplary embodiment with a rotated top-down view of a spinal insert. In the exemplary embodiment of FIG. 3a, rounded top portion 202 is shown as being substantially curved. Additionally, insert 200 may have slot 208. Slot 208 may extend from rounded top portion 202 to bottom portion 204. Additionally, another slot (not shown) may be disposed on an opposite side of insert 200 and may be disposed substantially opposite to slot 208. Further, in another exemplary embodiment the slot may be dispose diagonally across the insert 200 as is shown in FIG. 6c. The slots disposed on either side of insert 200 may allow for a device, such as a distractor, to have prong blades inserted through the slots of insert 200. When the prongs of a device are inserted into the slots, insert 200 may be positioned in any of a variety of fashions. Additionally, after insert 200 is inserted, for example into the spine of a person, the device inserted into the slots of insert 200 may be withdrawn without disturbing the location of insert 200 or any of the surrounding parts of the person.

FIGS. 3b and 3c show additional exemplary embodiments of inserts 214 and 226, respectively. In FIG. 3b, insert 214 is shown with rounded top portion 216 being substantially curved. Also the leading edge may be beveled or tapered to allow a little more ease of insertion.

Additionally, insert 214 may have insertion slot 224, which may extend from rounded top portion 216 to bottom portion 218. There may also be an insertion slot (not shown) disposed substantially opposite slot 224, similar to that in FIG. 3a. In FIG. 3c, insert 226 is shown with rounded top portion 228 being substantially curved. Additionally, insert 226 may have insertion slot 234, which may extend from rounded top portion 228 to bottom portion 230. There may also be an insertion slot (not shown) disposed substantially opposite slot 234, similar to that in FIG. 3a. Further, in another exemplary embodiment the slot may be dispose diagonally across the insert 200 as is shown in FIG. 6c, allowing enough room for the insert to pass by the slot 234.

In other exemplary embodiments of the invention, slots 208, 224 and 234 may be angled in any manner or positioned diagonally, longitudinally or horizontally, or any other orientation that would still enable one having ordinary skill in the art to properly insert the device, for example, in the posterior of the human spine. For example, slots 208, 224 and 234 may be angled so that they only occupy a portion of inserts 200, 214 and 226, respectively.

An exemplary rear view of a spinal insert is shown in FIG. 4. In this exemplary view, it may be seen that rear portions 204, 218 and 230 of inserts 200, 214 and 226, respectively, may appear substantially similar. In FIG. 4a, it is shown that insert 200 may have first and second insertion slots 208 and 210, respectively. These slots may be used in a similar manner to that described with respect to FIG. 3a. Additionally, insert 200 may have inserter hole 212. Inserter hole 212 may be used for a variety of purposes, for example, rod or inserter 103 may be inserted into the hole 212 to help guide the implant into position. Additionally, inserter hole 212 may be located in a central portion of insert 200, or may optionally be located on any location of bottom portion 204. Inserter hole 212 may also extend partially through insert 212, for example about 1-10 mm.

Similarly, in FIGS. 4b and 4c, it is shown that inserts 214 and 226 may also have first and second insertion slots. Insert 214 has first and second insertion slots 224 and 225, respectively, and insert 226 has first and second insertion slots 234 and 236, respectively. These slots may be used in a similar manner to that described with respect to FIG. 3a. Additionally, insert 214 may have inserter hole 223 and insert 226 may have inserter hole 238. Inserter holes 223 and 238 may be used for a variety of purposes, similar to those described with respect to FIG. 4a. Additionally, inserter holes 223 and 238 may be located in a central portion of inserts 214 or 226, respectively, or may optionally be located on any location of bottom portions 218 and 230, respectively. Inserter holes 223 and 238 may also extend partially through inserts 214 and 226, respectively, for example, about 1 to 10 mm.

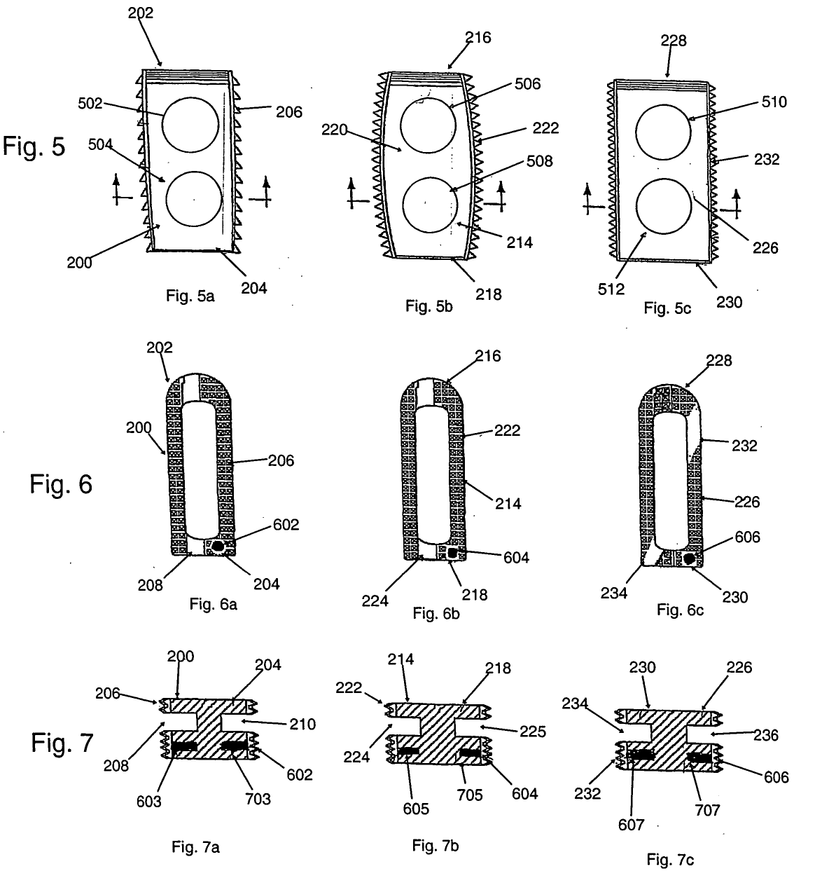

FIG. 5 shows another exemplary embodiment of an insert. In one exemplary embodiment shown in FIG. 5a, directional arrows are shown on either side of insert 200 to show an orientation of insert 200 when it can be inserted into the spine of a person. Additionally, holes 502 and 504 may drilled through insert 200 to allow one having ordinary skill in the art to evaluate bony fusion. For example the bone may not grow through these holes 502 and 504, but rather the bone will grow from knurl to knurl and 502 and 504 allow to act like windows to evaluate the fusion. Holes 502 and 504 may be formed so as to allow for evaluation of bone growth into insert 200. For example, after insert 200 is implanted in the spine of a person, vertebral bone growth maybe evaluated through holes 502 and 504. Likewise, the holes 502 and 504 may facilitate location of bone growth in post operative x-ray or other evaluation tool known to one having ordinary skill in the art. Similarly in FIG. 5b, tapered insert 214 has direction arrows showing one orientation of insert 214 when it may be inserted into the spine of a person. Additionally, holes 506 and 508 may be drilled through insert 214, and may provide a similar function as those discussed with respect to FIG. 5a. Further, in FIG. 5c, straight insert 226 has direction arrows showing one orientation of insert 226 when it may be inserted into the spine of a person. Additionally, holes 510 and 512 may be drilled through insert 226, and may provide a similar function as those discussed with respect to FIG. 5a. The holes 502, 506, and 510 may optionally be used for any other purpose known to one having ordinary skill in the art.

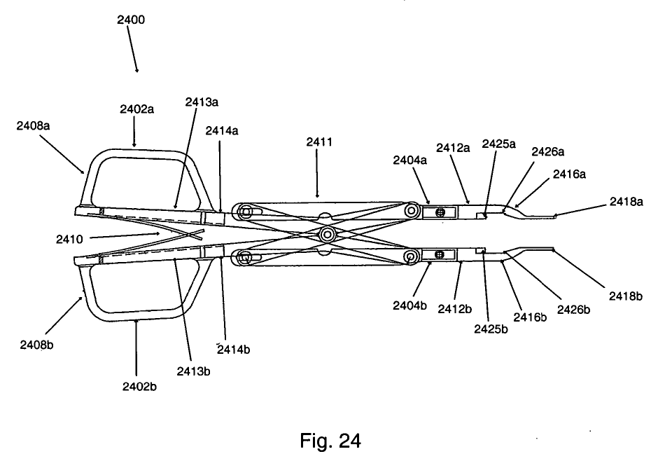

FIG. 6 shows additional exemplary embodiments of tapered insert 200, arched insert 214 and straight insert 226. The embodiments shown in FIGS. 6a-6c may be similar to those shown in FIGS. 3a-c. Slots 208, 224 and 234 of FIGS. 6a, 6b and 6c, respectively, may be disposed at an angle as in FIG. 6c to allow for the insertion of the distractor blades 2418a and 2418b (as shown in FIG. 24) which may aid in placing and orienting the spinal inserts. Additionally, inserts 200, 214 and 226 may have holes 602, 604 and 606, respectively, drilled into the inserts through the teeth or knurls 206. Holes 602, 604 and 606 may be drilled partially through the inserts and may act to receive a prong of a surgical instrument facilitate placement of the inserts. The holes 602, 604 and 606 generally replace the functionality of the inserter or rod holes of, for example in FIGS. 4a-4c. In another exemplary embodiment the holes 602, 604 and 606 may be drilled in the same plane as the inserter hole but instead of one inserter hole two or more inserter holes could be configured. Additionally, each of holes 602, 604 and 606 may have a hole drilled substantially on the opposite side of the insert. These holes, 603, 605 and 607, respectively, are shown in FIGS. 7a-c. In one exemplary embodiment, holes 602 and 603 of insert 200 may each have a prong of a medical device inserted into them. The prongs of the medical device may be used in a similar manner as surgical pliers, acting to hold insert 200 in place, move insert 200, or change the position or orientation of insert 200. After insert 200 is inserted into a spine of a person, the medical instrument may be removed from holes 602 and 603, and the medical instrument may be retracted or removed. Holes 604 and 605 of insert 214 and holes 606 and 607 of insert 226 may be used in a similar fashion as those described with respect to holes 602 and 603 of insert 200. Further the holes 602, 604 and 606 may be used in any way to facilitate placement of the insert 200 known to one having ordinary skill in the art.

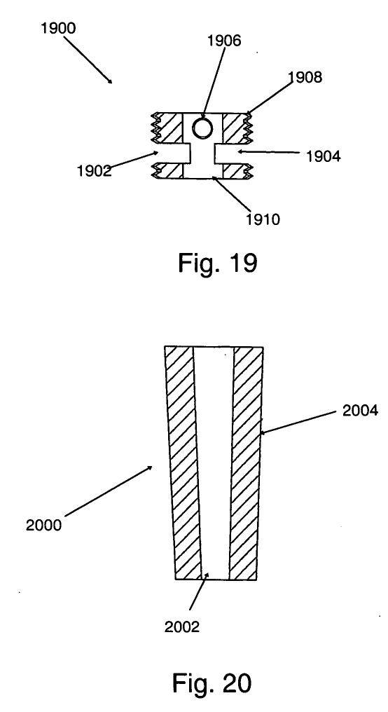

Generally referring to FIGS. 7a-c and 19-20 show further exemplary embodiments in rear views of the inserts described with respect to FIGS. 5 and 6. In these exemplary embodiments, insert 200 is shown with hole 602 on a top portion of the insert, insert 214 is shown with hole 604 on a top portion of the insert and insert 226 is shown with hole 606 on a top portion of the insert. Additionally, in FIG. 7a, interior portion 703 and teeth 206 may be formed out of different materials. For example, interior portion 703 (FIG. 7) or 1910 (FIG. 19) may be formed out of polyethylene or another polymer while teeth 206 or 1908 (FIG. 19) are formed of chrome molybdenum or another metallic substance. In other exemplary embodiments, interior portion 703 or 1910 may be polyurethane or polypropylene or poly ether ether keton (PEEK). Additionally, the teeth 206 or 1908 may be titanium or chromium molybdenum. Similarly, in FIGS. 7b and 7c, interior portions 705 and 707 may be formed out of polyethylene or another polymer while teeth 222 and 232 are formed of chrome molybdenum or another metallic substance. In other exemplary embodiments, interior portions 705 and 707 may be polyurethane, polypropylene or (poly ether ether ketone (PEEK)._Additionally, the teeth 206 may be titanium or chromium cobalt.

In another exemplary embodiment the insert may be engaged through the knurled surface with an pliers like inserter (not shown) as to prevent torsion while being inserted through the distractor, thereby demonstrating not only can the insert be engaged through the threaded slot but also through the sides with the knurl.

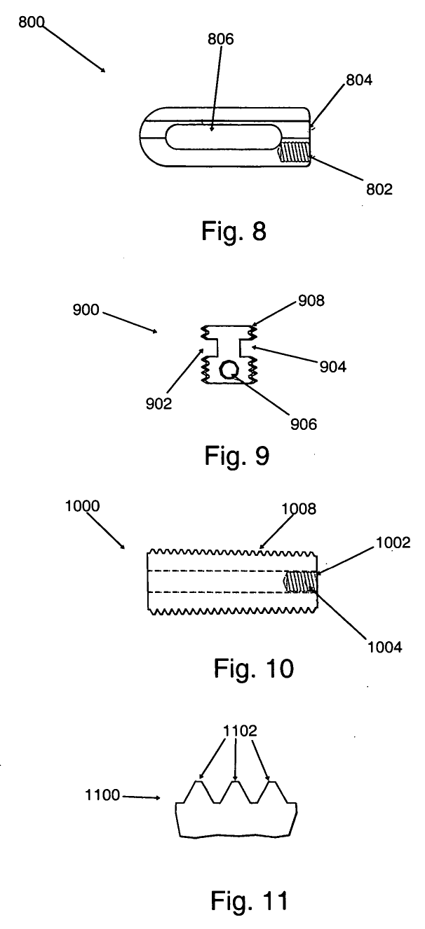

FIG. 8 shows another exemplary embodiment of an insert. Here, insert 800 may be a straight insert, such as that described with respect to FIG. 2c. In this embodiment, insert 800 may have inserter hole 802 and slot 804. Inserter 802 is shown as being disposed on a lower portion of insert 800. Additionally, inserter hole 802 is shown as extending from the rear portion of insert 800 to cut out area 806. Inserter hole 802 may also be threaded so as to receive a threaded insert, such as an inserter or rod 103. Also, slot 804 may extend the length of insert 800 and may be used similarly to the slots described above with respect to FIG. 3a. The slot may, optionally, be positioned diagonally across the insert 800 as is showing in FIG. 6c and provide a similar function.

A rotated view of an exemplary insert is shown in FIG. 9. Insert 900 may have slots 902 and 904. Slots 902 and 904 may allow for a device, such as a distractor 100, to have prongs inserted through slots 902 and 904. When the prongs of a device are inserted into slots 902 and 904, insert 900 may be positioned in any of a variety of fashions. Additionally, after insert 900 is inserted, for example into the spine of a person, the device inserted into the slots of insert 900 may be withdrawn without disturbing the location of insert 900 or any of the surrounding parts of the patient or patient's spine. Further, in FIG. 9, inserter hole 906 is shown. Inserter hole 906 may be located centrally in insert 900 or may alternatively be offset to either side of insert 900. Similar to insert hole 802 of FIG. 8, inserter hole 906 may extend part of the way through insert 900. Inserter hole 906 may be threaded, allowing for the insertion of a rod having threading, which may aid in placing, locating or orienting insert 900. Alternatively, any type of connection mechanism may used for the inserter hole 906 and the inserter or rod, such as using electromagnetism, magnets, clipping mechanism or tongue and groove type configuration or other surgical grade latching mechanism known to one having ordinary skill in the art. Further, teeth 908 may be disposed on either side of insert 900. Teeth 908 may be oriented directly out of insert 900, as shown in FIG. 9, or may be angled toward the top or bottom of insert 900. Additionally, teeth 908 may be disposed on either side of insert 900 as well as above and below slots 902 and 904.

Another exemplary rotated view of an insert is shown in FIG. 10. In this cutout view, insert 1000 may have screw 1004 inserted into inserter hole 1002. Screw 1004 may only penetrate insert 1000 a short distance, as shown in FIG. 10. In other exemplary embodiments, rod 1004 may penetrate insert 1000 to different depths. Additionally, teeth 1008 are shown as projecting straight out of insert 1000 in this exemplary embodiment. In other exemplary embodiments, teeth 1008 may be in any of a variety of different orientations.

FIG. 11 shows an exemplary close up view of knurls or teeth 1102 of insert 1100. In this embodiment, the knurls or teeth 1102 are substantially triangular. Teeth 1102 may act to grip a surrounding surface and prevent movement of insert 1100. In other exemplary embodiments, teeth 1102 may be angled differently, such as towards the left or the right. Additionally teeth 1102 may be formed out of any other shape known to one having ordinary skill in that art that would provide adequate grip against any of a variety of surfaces, such as bone or tissue, so as to prevent the movement of insert 1100 after insert 1100 is inserted.

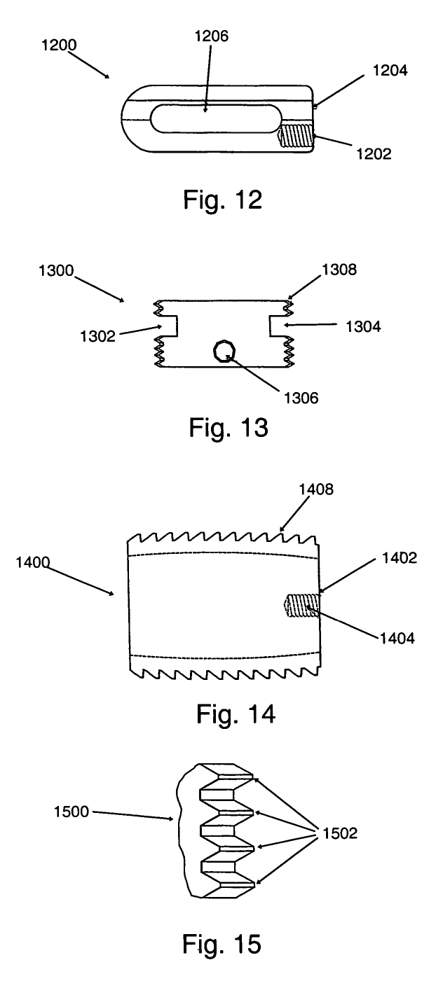

FIG. 12 shows yet another exemplary embodiment of an insert. Here, insert 1200 may be a tapered insert, similar to that described in FIG. 2a. In this embodiment, insert 1200 may have inserter hole 1202 and slot 1204. Inserter 1202 is shown as being disposed on a lower portion of insert 1200. Additionally, inserter hole 1202 is shown as extending from the rear portion of insert 1200 to cut out area 1206. Inserter hole 1202 may also be threaded so as to receive a threaded insert, such as a screw. Also, slot 1204 may extend the length of insert 1200 and may be used similarly to the slots described above with respect to FIG. 3a. Another slot may be disposed opposite to slot 1204 and provide a similar function.

A rotated view of an exemplary insert is shown in FIG. 13. Insert 1300 may have slots 1302 and 1304. Slots 1302 and 1304 may allow for a device, such as a distractor, to have prongs inserted through slots 1302 and 1304. When the prongs of a device are inserted into slots 1302 and 1304, insert 1300 may be positioned in any of a variety of fashions. Additionally, after insert 1300 is inserted, for example into the spine of a person, the device inserted into the slots of insert 1300 may be withdrawn without disturbing the location of insert 1300 or any of the surrounding parts of the person. Further, in FIG. 13, inserter hole 1306 is shown. Inserter hole 1306 may be located centrally in insert 1300 or may alternatively be offset to either side of insert 1300. Similar to insert hole 1202 of FIG. 12, inserter hole 1306 may extend part of the way through insert 1300. Further, teeth 1308 may be disposed on either side of insert 1300. Teeth 1308 may be oriented directly out of insert 1300, as shown in FIG. 13, or may be angled toward the top or bottom of insert 1300. Additionally, teeth 1308 may be disposed on either side of insert 1300 as well as above and below slots 1302 and 1304. Additionally, it may be noted that insert 1300 is shown as wider than insert 900 of FIG. 9. These inserts may be formed in any of a variety of sizes and shapes known to one having ordinary skill in the art. Additionally, the sizes and widths of the inserts may be varied or tailored to suit a particular need. For example, larger inserts may be utilized in situations where there are larger gaps between vertebrae or where greater separation between vertebrae is desired. Similarly, smaller inserts may be used where smaller gaps exist or where smaller separation is desired or may be achieved.

Another exemplary rotated view of an insert is shown in FIG. 14. In this cutout view, insert 1400 may have screw 1404 inserted into threaded inserter hole 1402. Screw 1404 may only penetrate insert 1400 a short distance, as shown in FIG. 14. In other exemplary embodiments, screw 1404 may penetrate insert 1400 to different depths. Additionally, teeth 1408 are shown as projecting towards the left portion of insert 1400 in this exemplary embodiment. In other exemplary embodiments, teeth 1408 may be in any of a variety of different orientations, such as projecting at different angles or projecting straight out of insert 1400.

FIG. 15 shows an exemplary close up view of teeth 1502 of insert 1500. In this embodiment, teeth 1502 are substantially triangular. Teeth 1502 may act to grip a surrounding surface and prevent movement of insert 1500. In other exemplary embodiments, teeth 1502 may be angled differently, such as towards the left or the right. Additionally teeth 1502 may be formed out of any other shape known to one having ordinary skill in that art that would provide adequate grip against any of a variety of surfaces, such as bone or tissue, so as to prevent the movement of insert 1500 after insert 1500 is inserted.

Another exemplary rotated and cutout view of an insert is shown in FIG. 16. In this cutout view, insert 1600 may be formed having an arched shape, similar to that described with respect to FIG. 2b. This insert would be ideal for a spine configuration as is shown, for example, in FIG. 33. Where the width of end points x, y, of the spine are substantially equal. The center portion z may be substantially wider than x and y. Likewise, insert 1600 may have end points x, y, that are equal where the center point z is substantially wider to accommodate the space 3304 in the cross-sectional spine shown in FIG. 34. With further progression of spondylosis a scalloped appearance may occur in the disc space relative to the adjacent vertebral bodies. In order to promote bony fusion or maintain spinal dynamics utmost anatomical contact between the knurled surfaces of the implant is imperative in order to overcome this spinal defect. Thus, in this exemplary embodiment, the center portion of insert 1600 may be substantially wider than either distal end of insert 1600. This may provide a better fit between the vertebrae of some subjects. Additionally, insert 1600 may have screw 1604 inserted into threaded inserter hole 1602. Screw 1604 may only penetrate insert 1600 a short distance, as shown in FIG. 16. In other exemplary embodiments, screw 1604 may penetrate insert 1600 to different depths. Additionally, teeth 1608 are shown as projecting straight out of insert 1600 in this exemplary embodiment. In other exemplary embodiments, teeth 1608 may be in any of a variety of different orientations.

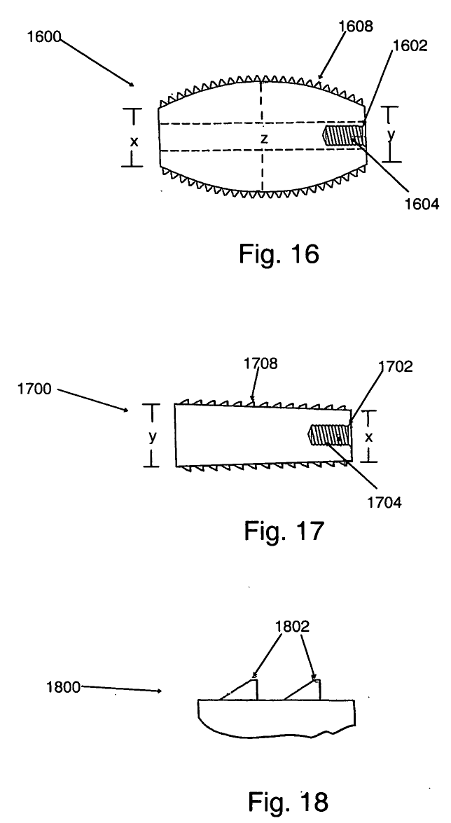

Yet another exemplary rotated and cutout view of an insert is shown in FIG. 17. In this cutout view, insert 1700 may be formed having a tapered shape, similar to that described with respect to FIG. 2a. Thus, in this exemplary embodiment, the one distal end of insert 1700 may be substantially wider than another distal end of insert 1700. This may provide a better fit between the vertebrae of some subjects and provide different insertion characteristics than inserts having other shapes. This insert would be ideal for a spine configuration as is shown, for example, in FIG. 32 where the width of end point y 3202 is substantially 2 times as wide as end point x 3204 in one embodiment. Also, insert 1700 may have screw 1704 inserted into threaded inserter hole 1702. Screw 1704 may only penetrate insert 1700 a short distance, as shown in FIG. 17. In other exemplary embodiments, screw 1704 may penetrate insert 1700 to different depths. Additionally, teeth 1708 are shown as projecting towards the left portion of insert 1700 in this exemplary embodiment. In other exemplary embodiments, teeth 1708 may be in any of a variety of different orientations, such as projecting at different angles or projecting straight out of insert 1700. With the progression of spondylosis or in post surgical patients, patients may lose their natural curvature of their spine. The loss of the lumbar lordosis leads to a poor sagittal balance. This implant is geared for not only to allow greater contact between implant and cortical bone, but also to restore sagittal balance in those patient who have lost it due to degenerative or post surgical reasons.

Additionally, with respect to FIGS. 10-17, various sizes and shapes of inserts are shown. Each of the inserts, depending on its size or shape, as well as other properties, such as the orientation of the teeth projecting from the body of the insert, may be utilized in any of a variety of situations or circumstances. Some embodiments may provide better fitment in certain circumstances and other embodiments may provide for easier insertion or removal. Still other embodiments may better limit or prevent movement in certain circumstances. Therefore, any of the exemplary embodiments shown or described herein may be used in any of a variety of different circumstances.

FIG. 18 shows an exemplary close up view of teeth 1802 of insert 1800. In this embodiment, teeth 1802 are substantially triangular and are angled to the right. Teeth 1802 may act to grip a surrounding surface and prevent movement of insert 1800. In other exemplary embodiments, teeth 1802 may be angled differently, such as towards the left or the right. Additionally teeth 1802 may be formed out of any other shape known to one having ordinary skill in that art that would provide adequate grip against any of a variety of surfaces, such as bone or tissue, so as to prevent the movement of insert 1800 after insert 1800 is inserted.

A rotated view of an exemplary insert is shown in FIG. 19. Insert 1900 may have slots 1902 and 1904. Slots 1902 and 1904 may allow for a device, such as a distractor 100, to have prongs inserted through slots 1902 and 1904. When the prongs of a device are inserted into slots 1902 and 1904, insert 1900 may be positioned in any of a variety of fashions. Additionally, after insert 1900 is inserted, for example into the spine of a person, the device inserted into the slots of insert 1900 may be withdrawn without disturbing the location of insert 1900 or any of the surrounding parts of the person. Further, in FIG. 19, inserter hole 1906 is shown. Inserter hole 1906 may be located centrally in insert 1900 or may alternatively be offset to either side of insert 1900. Also, inserter hole 1906 may be located at either a top or a bottom portion of insert 1900. Further, similar to inserter holes 1602 or 1702 of FIGS. 16 and 17, respectively, inserter hole 1906 may extend part of the way through insert 1900. Further, teeth 1908 may be disposed on either side of insert 1900. Teeth 1908 may be oriented directly out of insert 1900, as shown in FIG. 19, or may be angled toward the top or bottom of insert 1900. Additionally, teeth 1908 may be disposed on either side of insert 1900 as well as above and below slots 1902 and 1904. In order to maintain or restore natural spinal dynamics the goal centers on the ability to greatly on the dynamic insert to anatomically articulate with the cortical bone of the two adjacent vertebral bodies. If there is a loss of this contact, then with natural spinal motion or dynamics the implant may migrate and cause neurological injury. Therefore posterior artificial dynamic inserts will require a technique to overcome the smaller height of the posterior disc space and allow the surgeon to implant an insert which will not only restore sagittal balance but also to prevent migration of the implant.

FIGS. 19 and 20 further show another exemplary embodiment of an insert. In these embodiment, a top-down, cutout version of a tapered insert, similar to that described with respect to FIG. 17, is shown. Here insert 2000 has a center portion 2002. Additionally, in FIGS. 19 and 20, interior portion 1910 and 2004 teeth 1908 may be formed out of different materials. For example, interior portion 1910 or 1910 (FIG. 19) may be formed out of polyethylene or another polymer while teeth 206 or 1908 (FIG. 19) are formed of chrome molybdenum or another metallic substance. In other exemplary embodiments, interior portion 1910 or 2002 may be polyurethane or polypropylene to flex in a manner known to one having skill in the art that would be similar to a disk in a human spine. Additionally, the teeth 1908 may be titanium, chromium or molybdenum or any other surgical grade material known to one having ordinary skill in the art to prevent migration within the disc space.

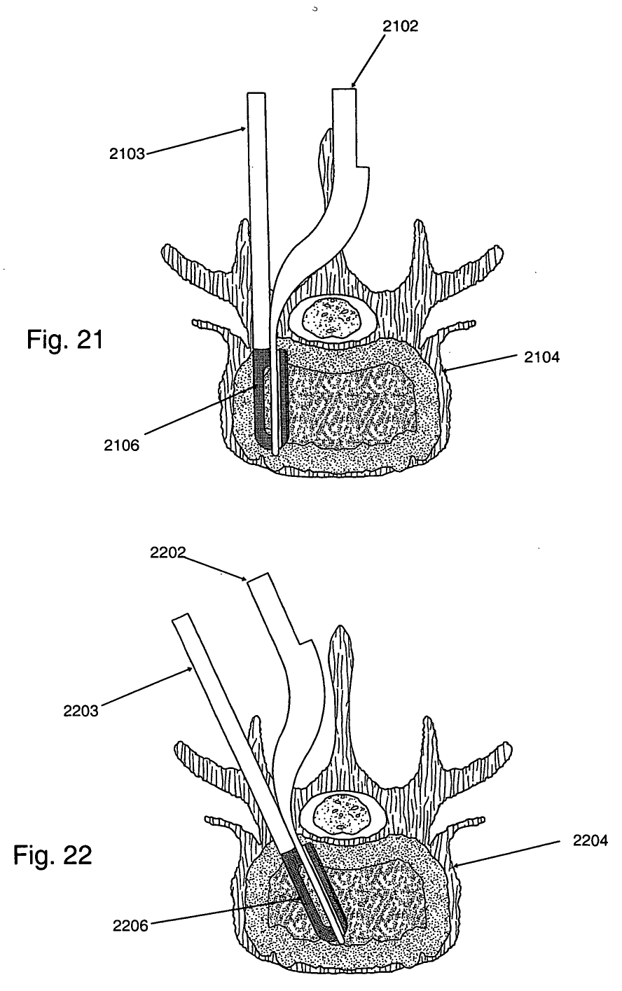

An exemplary embodiment of a spinal insert being inserted is shown in FIG. 21. In this embodiment, distractor 2102 is shown at an approximately 90 degree angle from spinal column 2104. Additionally, rod 2103 is shown as holding insert 2106. Rod 2103 may be engaged with insert 2106 in any of a variety of manners. In one exemplary embodiment, rod 2103 has threading at a distal end that may be inserted into a threaded hole in insert 2106. Insert 2106 may be, for example, one of any of the different types of inserts discussed herein. Also, distractor 2102 is shown as utilizing slots disposed on either side of insert 2106. These slots may be similar to those discussed with respect to FIG. 3a. After insert 2106 is placed, distractor 2102 and rod 2103 may be retracted and removed.

Another exemplary embodiment of a spinal insert being inserted is shown in FIG. 22. Here, distractor 2202 is shown at an approximately 45 degree angle from spinal column 2204. The distractor 2202 may optionally be placed in a 45 degree angle plane, another embodiment could be the distractor 2202 is still in the vertical plane like FIG. 21 but the tips 2418a, 2418b are angled 45 degrees. Since these tips could be made of titanium, steel, carbon graphite, ceramic, PEEK or any other material know to one having ordinary skill in the art and could be disposable to the point if there is a overt fracture or microfractures with in the tips then the whole distractor 2202 will not need to be replaced. In further embodiments, a distractor may be inserted into a spinal column at any angle that allows for the insertion of a spinal insert. Additionally, rod 2203 is shown as holding insert 2206. Rod 2203 may be engaged with insert 2206 in any of a variety of manners. In one exemplary embodiment, rod 2203 has threading at a distal end that may be inserted into a threaded hole in insert 2206. Also, in FIG. 22, insert 2206 may be, for example, one of any of the different types of inserts discussed herein. Also, distractor 2202 is shown as utilizing slots disposed on either side of insert 2206. These slots may be similar to those discussed with respect to FIG. 3a. Similar to the exemplary embodiment shown in FIG. 21, both distractor 2202 and rod 2203 may be retracted and removed after insert 2206 is placed.

Additionally, with respect to FIGS. 21 and 22, distractor 2102/2202 may be hinged. As shown in FIG. 22, a hinge 2210 may be disposed on distractor 2202. Hinge 2210 can allow for a user of distractor 2202 to move a portion of distractor 2202 out of a line of sight. Hinge 2210 may also be two hinges, for example, a first hinge disposed on a first jaw of a distractor and a second hinge disposed on a second jaw of a distractor. For example, if a user has inserted distractor 2202 into spinal column 2204, distractor 2202 may be oriented in such as fashion that it may block some or all of a user's view of insert 2206. The use of hinge 2210 may therefore allow a user to move a portion of distractor 2202 out of the line of sight and therefore aid in the positioning and orienting of insert 2206. Hinge 2210 may be disposed in any of a variety of different locations on distractor 2202, depending on the different size of distractor being used, as well as depending on where a user would desire to hinge a distractor for a specific use or operation.

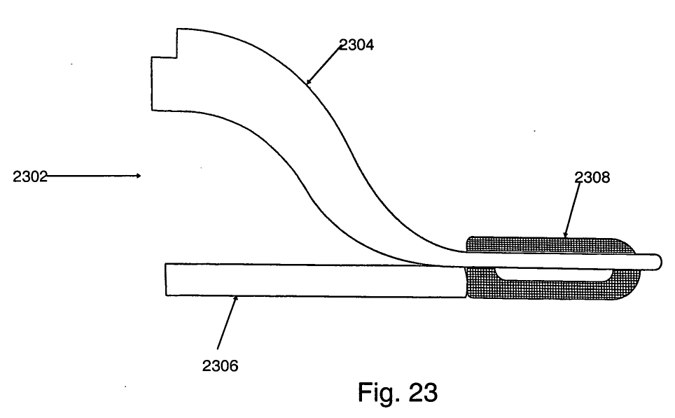

An exemplary view of a distractor is shown in FIG. 23. In this embodiment, distractor 2302 may have a variety of parts and components. Arm 2304 may have an angled portion that terminates in a pair of blades. The slope of arm 2304 may be such that it allows a user to better position distractor 2302 for disc insertion and removal, for example. Additionally, the blades of arm 2304 may be configured to clasp and hold a spinal insert, such as spinal insert 2308. Arm 2304 may be formed in any of a variety of manners and out of any of a variety of materials known to one having ordinary skill in the art, such as steel or other materials as described above or known to one having ordinary skill in the art. Additionally, arm 2304 may be formed in different sizes and shapes so as to be able to grasp, clasp or hold any of a variety of different sized spinal inserts. Distractor 2302 may utilize rod 2306. Rod 2306 may be formed and made in any manner known to one having ordinary skill in the art. Additionally 2306 may have a threaded distal end that may be inserted into spinal insert 2308. Thus, rod 2306 may be securely screwed into insert 2308 and may also be unscrewed and removed from insert 2308. Further, rod 2306 may be used for any of a variety of functions, such as positioning insert 2308, securing insert 2308 or stabilizing distractor 2302. In a further embodiment, distractor 2302 may hold insert 2308 and be positioned in any of a variety of manners and at any angle so as to allow a user to insert a spinal insert between vertebrae.

FIG. 24 shows an exemplary embodiment of a distractor. Distractor 2400 may have a pair of handles 2402a and 2402b, which may be movable with respect to each other to actuate a pair of jaws 2404a and 24044b coupled thereto. Distractor 2400 may be used for a variety of procedures, for example spinal disc distraction and spinal implant or insert insertion. Distractor 2400 may therefore be configured such that actuation of handles 12 (12a, 12b) moves jaws 14 (14a, 14b) apart substantially parallel along a distraction axis to a working position corresponding to the desired resulting relative position of the endplates. For example, the blades may be moved to a substantially parallel position to separate adjacent vertebrae to be treated.

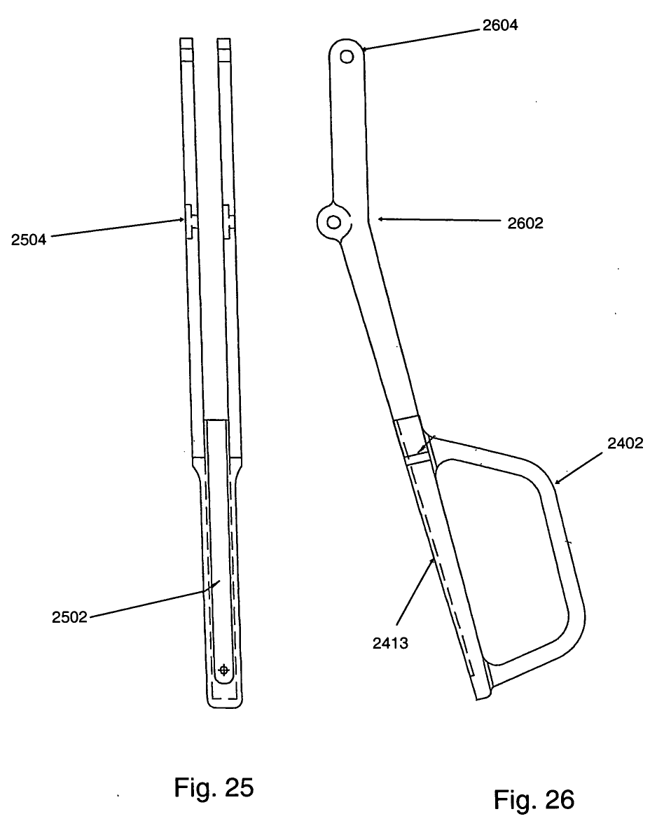

Generally referring to FIGS. 25 and 26, another attendant advantage is the ability to grasp the handle 2402 to prevent the distractor from moving down towards the ground if, for example a surgeon's latex glove was to slip cause neurologic injury. The distractor handle 2502, 2402 has a hand placement area which acts as a safety mechanism to prevent sudden motions when applying the distraction force. Further the distractor may have a bend 2602 and a distal connection point allowing hinged movement. Likewise the connection point 2504 facilitates hinged movement. The connection points 2504, 2602 and 2604 may be any kind of connection that allows in hinged movement that is known to one having ordinary skill in the art.

Further, as shown in FIG. 24, handles 2402 and jaws 2404 may be configured to move jaws 2404 apart along a distraction axis a sufficient amount to adequately separate adjacent vertebrae to be treated (for example 5 mm-33 mm, or typically 13 mm-15 mm) yet to occupy a minimal amount of space within the insertion region during the procedure. Thus, handles 2402 and jaws 2404 may be pivotally coupled together in a scissors configuration such that movement of handles 2404a and 2404b together causes jaws 2404a and 2404b to move apart and effect insertion or distraction of object or organic material between which jaws 2404 are positioned. Thus, proximal ends 2408a and 2408b of handles 2402 may be configured to facilitate gripping.

In addition, distractor 2400 may have biasing element 2410, such as a pair of leaf springs, which may maintain handles 2402a and 2402b in a spaced-apart configuration such that jaws 2404a and 2404b may be close together, ready for insertion through a small incision and narrow passage through the patient in the neutral configuration of FIG. 24.

Further, distractor mechanism 2411 may be provided such that movement of handles 2402 to actuate distractor mechanism 2411 can cause jaws 2404 to move apart to effect distraction of adjacent elements such as vertebrae. Distractor mechanism 2411 may have a scissor-type configuration such that handle 2402a and jaw 2404a are at opposite ends of a first lever arm and handle 2402b and jaw 2404b are on opposite ends of a second lever arm pivotally coupled to the first lever arm. Additionally, distractor mechanism 2411 may be in the form of a triple-acting scissor configuration having greater than one pivot point, for example three pivot points, thus reducing the amount of space required along a distraction axis and laterally away from a distractor mechanism longitudinal axis to effectuate distraction. Also, in order to form a triple-acting scissor configuration, handles 2402 and jaws 2404 can be provided on separate lever arms which are pivotally coupled together. In particular, handle 2404a can be formed at a proximal end of proximal lever arm 2413a, handle 2402b is formed at a proximal end of lever arm 2413b, jaw 2404a is formed at a distal end of distal lever arm 2412a, and jaw 2404b is formed at a distal end of distal lever arm 2413b. Distal end 2414a of proximal lever arm 2413a is pivotally coupled to proximal end of distal lever arm 2412a and distal end 2414b of proximal lever arm 2413b is pivotally coupled to a proximal end of distal lever arm 2412b. In order to actuate the triple-acting mechanism to effectuate distraction and hence movement of jaws 2404a and 2404b apart upon movement of handles 2402a and 2402b together, one set of lever arms can be laterally pivotally coupled together and the other set of lever arms is crosswise pivotally coupled together. Further, with distractor mechanism 2411, the triple-acting configuration can break the pivoting action into three components, reducing the total movement of distractor mechanism 2411 required along a distraction axis.

An additional feature of distractor 2411 which can facilitate use thereof during distraction is the relative offset positions of jaws 2404a and 2404b, handles 2402a and 2402b, and distractor mechanism 2411 with respect to one another, as may be appreciated in the side elevational view of FIG. 26. In a further embodiment, distal jaw ends 2416a and 2416b may be positioned to properly distract adjacent vertebrae and distractor mechanism 2411 and handles 2402a and 2402b may be offset relative to distal jaw ends 2416a and 2416b to permit optimal visualization of distal jaw ends 2416a and 2416b from the proximal end of distractor 2400 (outside the patient's body) during distraction. For example, a distal bend may be provided immediately proximal of distal jaw ends 2416a and 2416b, as may be appreciated with reference to FIG. 26. Thus, the remainder of distractor 2400 (i.e., the proximal portions of distractor 2400 such as distractor mechanism 2411 and handles 2402a and 2402b) may be in a different plane from the plane of distal jaw ends 2416a and 2416b and the distraction site. With such an offset, visualization of the distraction site and of insertion of the implant or insert therein is enhanced. Also, such offset of portions of distractor 2400, such as distractor mechanism 2411 and handles 2402a and 2402b, from the distal jaw ends 2416 accommodate an implant holder for insertion of the implant to permit a substantially straight insertion of the implant holder. The bend proximate distal jaw ends 2416 may be between 0 degrees and 30 degrees, for example 10 degrees, to achieve improved visualization and increased area for the implant holder.

In one exemplary embodiment the sharp edges 2426a, 2426b may be configured to be blade guides to start an insert 102 or those shown in FIGS. 2a-2c, to be guided smoothly into a distracted disc space to facilitate placement of an insert, for example, in in posterior lumbar surgery.

A distractor 2400 provided in accordance with the principles of the present invention is configured to distract adjacent vertebrae so that an implant may be inserted therebetween. Preferably, each jaw of a distractor formed in accordance with the principles of the present invention is provided with a blade, such as blades 2418a and 2418b, shaped and configured to contact a vertebral endplate and also to permit insertion of an implant there between via a parallel opening movement of the blades 2418a and 2418b which provides optimal space between the disc space. The parallel opening movement of the blades 2418a and 2418b may provide an optimal opening for a surgeon to safely and efficiently insert an implant. The parallel opening mechanism may decrease risk of injury and make posterior spinal surgery more efficient and safe to both patient and surgeon. Once the implant is properly positioned between the vertebral endplates, the distractor, along with its blades, may be removed from the distraction site in the patient.

As shown in FIG. 24, blades 2418a and 2418b are provided on jaws 2404a and 2404b, respectively, to engage the vertebrae to be distracted. In a preferred embodiment, blades 2418a and 2418b may be configured and shaped to correspond to a slot in an insert, such as those discussed in earlier embodiments. Thus, as the selected implant is moved toward the treatment site with a desired insertion tool, insert contacting surfaces of blades 2418a and 2418b contact respective slots in the inserts. Additionally, the insert contacting surfaces of blades 2418a and 2418b may be closer together than the point of connection of blades 2418a and 2418b to respective jaws 2404a and 2404b. Thus, jaws 2404a and 2404b may be sufficiently spaced apart to permit insertion of the thickest dimension of the implant therebetween, yet blades 2418a and 2418b can be closer together to account for the narrower dimension of the implant in the region of slots on an insert and thereby to securely grasp the implant via the slots.

Blades 2418 may converge directly towards each other in a distal direction before actuation of distractor mechanism 2411 as may be appreciated with reference to FIG. 24. Thus, upon actuation of distractor mechanism 2411 and pivoting apart of jaws 2404, blades 2418, and particularly the outwardly facing distracting surfaces of the blades (positioned to contact the endplates in the treatment site), may be moved into an orientation appropriate for the vertebral region being treated. For example, actuation of distractor mechanism 2411 may move the distracting surfaces of blades 2418 into a parallel orientation with respect to each other (i.e. moving blades 2418 apart while maintaining a 180 degree angle between blades 2418a and 2418b) to securely engage endplates which are parallel with respect to each other.

FIGS. 25 and 26 show further exemplary embodiments of a distractor. Specifically, FIGS. 25 and 26 show separated components of distractor 2400.

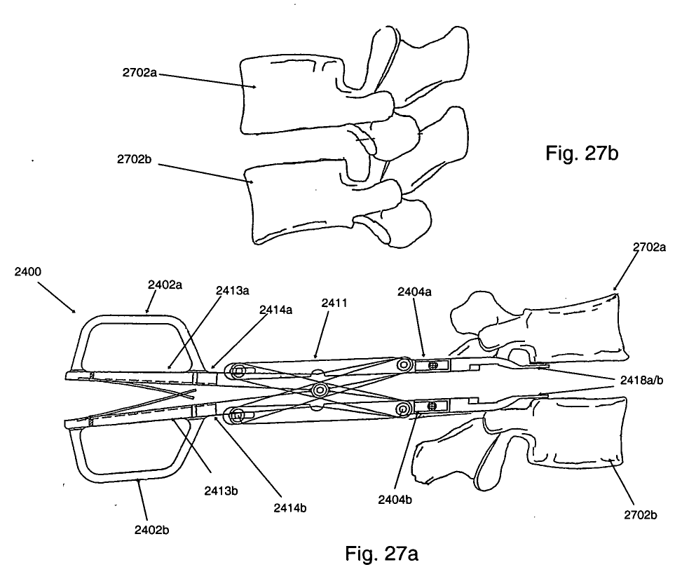

FIG. 27 shows an exemplary embodiment of a distractor being used. In this embodiment, distractor 2400 is shown as inserted into vertebra 2702. Handles 2402 may be used to actuate jaws 2404, thus separating blades 2418a and 2418b. The contact portions of blades 2418a and 2418b contact upper vertebra portion 2702a and lower vertebra portion 2702b, respectively, and can act to separate upper and lower vertebra 2702a and 2702b. This separation may allow for the insertion of a spinal insert or implant.

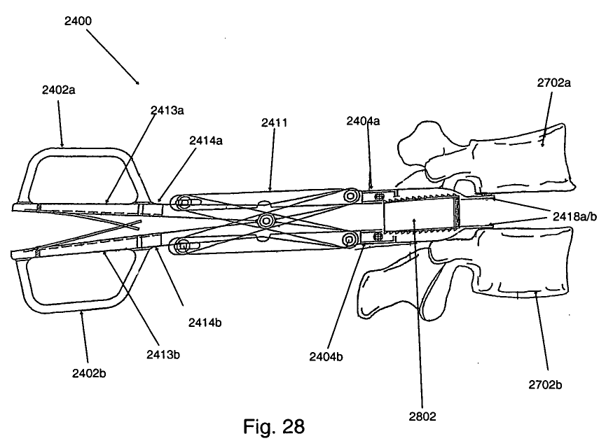

FIG. 28 shows another exemplary embodiment of a distractor being used. Similar to FIG. 27, distractor 2400 is shown as inserted into vertebra 2702, providing separation between upper vertebra portion 2702a and 2702b. Additionally, in this embodiment, insert 2802 is shown as being used with distractor 2400. Insert 2802 may be any type of insert, for example any type of insert described herein. Distractor 2400 may hold insert 2802 between jaws 2404a and 2404b until there is sufficient separation between upper vertebra portion 2702a and 2702b so as to insert spinal insert 2802 between upper vertebra portion 2702a and lower vertebra portion 2702b. Insert 2802 may have hole that accepts a rod as an insert, allowing for insert 2802 to be placed between upper vertebra portion 2702a and lower vertebra portion 2702b and aiding with the withdrawal of distractor 2400 there from.

FIG. 28 further shows the benefits of the parallel opening movement of the blades 2418a, 2418b which moves the vertebral bones 2702a and 2702b apart so that no grooves or canals are formed in the bone near the distractor blades 2418a, 2418b when inserting the device which greatly reduces the chances of migration once the insert 2802 is implanted and the distractor 2400 removed. This technique is safer for the surgeon and will create superior results for the patient. For example, this parallel distraction technique and system of implanting an insert reduces the chance of a surgeon pushing the insert into the bone of a patient with soft vertebrae because the space created with the parallel distraction technique made available by the distractor 2400 opens the disc space optimally for insertion of an insert such as the tapered cage 2802. Likewise, this technique facilitates trials during surgery by reducing the surgical impact in the vertebrae 2702a and 2702b with less scratching and scarring of the bone surface. For example, during surgery, many different types of implants, tapered 200, arched or curved 216 or straight 228 may be inserted into the disc space and an x-ray taken to determine which insert will provide optimal results for the patient based on the space between the patient's vertebrae. For example, some patient's disc space may be contoured as in FIG. 33 which would require an arched insert 216. Likewise others as shown, for example, in FIG. 32 would benefit from a tapered insert 200.

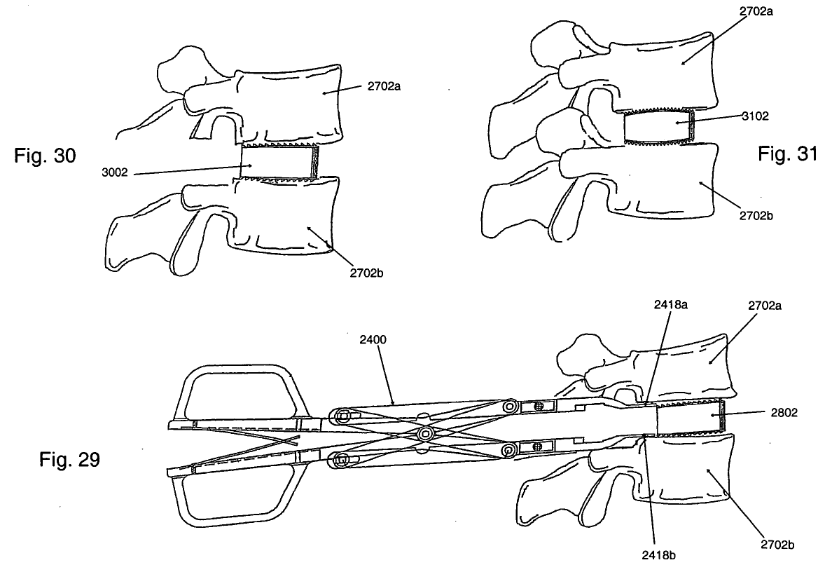

FIG. 29 shows a further embodiment of a distractor being used. In this embodiment, insert 2802 has been placed between upper vertebra portion 2702a and lower vertebra portion 2702b. Blades 2418a and 2418b have been partially retracted from upper vertebra portion 2702a and lower vertebra portion 2702b and the lower contact portion of blades 2418a and 2418b are no long in contact with insert 2802. Additionally, if a rod is used to help position and insert spinal insert 2802, it may also be retracted after insert 2802 is placed between upper vertebra portion 2702a and lower vertebra portion 2702b.

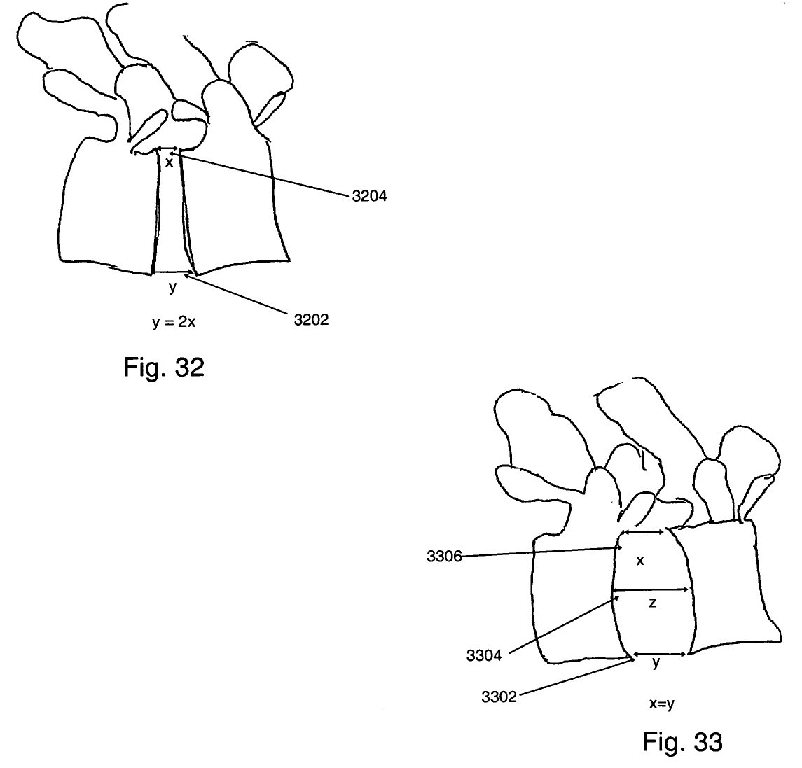

FIGS. 30 and 31 show exemplary embodiments of spinal inserts that may be inserted into a spinal column. In FIG. 30, tapered insert 3002 has been inserted between upper vertebra portion 2702a and lower vertebra portion 2702b. In FIG. 31, arched or curved insert 3102 has been inserted between upper vertebra portion 2702a and lower vertebra portion 2702b. Each of these inserts may be placed with the assistance of distractor 2400 in a manner similar to that described above with respect to FIGS. 27 and 28. Additionally, each insert 3002 and 3102 may provide different advantages when inserted. For example, tapered insert 3002 may be placed in such a manner where a non-tapered insert would not fit. Additionally, tapered insert 3002 or arched insert 3102 may be used in situations where the vertebra are shaped in such a manner that would not otherwise accept an insert, or where tapered insert 3002 or arched insert 3102 may provide an improved fit.

Curved Cage 3102 with further progression of spondylosis a scalloped appearance may occur in the disc space relative to the adjacent vertebral bodies. In order to promote bony fusion or maintain spinal dynamics utmost anatomical contact between the knurled surfaces of the implant 3102 in at least one exemplary embodiment may assist to overcome this spinal defect.

Tapered Cage 3002 with the progression of spondylosis or in post surgical patients, patients may lose their natural curvature of their spine. The loss of the lumbar lordosis leads to a poor sagittal balance. The tapered cage 3002 is geared for not only to allow greater contact between implant 3002 and cortical bone, but also to restore sagittal balance in those patient who have lost it due to degenerative or post surgical.

The foregoing description and accompanying drawings illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed:

1. A spinal insert, comprising:
a first portion with a slot and a surface adapted to engage a contacting surface;
a second portion with a slot and a surface adapted to engage a contacting surface;
a third portion that is substantially flat;
a fourth portion that is substantially flat;
a fifth portion that is substantially flat;
a sixth portion that is substantially curved; and
a central body.

2. The spinal insert of claim 1, wherein the surface adapted to engage a contacting surface in the first portion and the surface adapted to engage a contacting surface in the second portion are outward projecting knurlings.

3. The spinal insert of claim 1, wherein the slot disposed on the first portion and the slot disposed on the second portion are designed to receive a surgical tool.

4. The spinal insert of claim 1, wherein the slot disposed on the first portion and the slot disposed on the second portion run the length of the first portion and the second portion, respectively.

5. The spinal insert of claim 1, wherein the slot disposed on the first portion and the slot disposed on the second portion run at an angle on the first portion and the second portion, respectively.

6. The spinal insert of claim 5, wherein the angle of the slot on the first portion is the same as the angle of the slot on the second portion.

7. The spinal insert of claim 1, wherein the first portion and the second portion each have receiving holes designed to receive a surgical tool.

8. The spinal insert of claim 1, wherein the fifth portion has a receiving hole designed to receive a surgical tool.

9. The spinal insert of claim 8, wherein the receiving hole is offset from the center of the fifth portion.

10. The spinal insert of claim 1, wherein the first portion and the second portion are substantially flat.

11. The spinal insert of claim 1, wherein the first portion and the second portion are substantially tapered from a proximal end to a distal end.

12. The spinal insert of claim 1, wherein the first portion and the second portion are substantially arched in the center.

13. The spinal insert of claim 1, wherein the first portion and the second portion are formed of a different material than the central body.

14. The spinal insert of claim 13, wherein the first portion and the second portion are formed of chrome molybdenum and the central body is formed of polyethylene.

15. The spinal insert of claim 1, wherein the sixth portion is substantially curved.

16. A distractor, comprising:
a first handle;
a first jaw extending from the first handle, wherein at least a portion of the first jaw extends at an angle with respect to the first handle;
a second handle;
a second jaw extending from the second handle, wherein at least a portion of the second jaw extends at an angle with respect to the second handle; and
a distractor mechanism coupled between the first handle and the first jaw and the second handle and the second jaw such that movement of the first handle and the second handle toward each other actuates said distractor mechanism to move the entire length of the first jaw apart from the entire length of the second jaw in parallel.

17. The distractor of claim 16, further comprising:
a first blade with at least one sharp edge extending from the first jaw; and
a second blade with at least one sharp edge extending from the second jaw.

18. The distractor of claim 17, wherein the entire length of the first blade is parallel to the entire length of the second blade when the first jaw is moved apart from the second jaw.

19. The distractor of claim 17, wherein the first blade and the second blade are disposed on a different plane than the first jaw and the second jaw, respectively.

20. The distractor of claim 17, wherein the first blade and the second blade are configured to fit into slots disposed on a first portion and a second portion of a spinal insert.

21. The distractor of claim 16, further comprising a hinge disposed on the first and second jaws.

22. The distractor of claim 16, wherein the hinge on the first and second jaws allows for the first and second handles and a first portion of the first and second jaws to be angled away from a second portion of the first and second jaws.

23. The distractor of claim 16, wherein there is a bend disposed between the first and second handles and the first and second jaws.

24. The distractor of claim 16, further comprising a biasing element disposed between the first handle and the second handle.

25. A system for implanting a spinal insert, comprising:
clasping a spinal insert with a distractor;
inserting a rod into a in the spinal insert;
separating two vertebrae using two blades on the distractor;
inserting the distractor and the spinal insert between two vertebrae;
placing the spinal insert between the two vertebrae;
removing the rod from the spinal insert;
retracting the two vertebrae with the distractor;
allowing the vertebrae to contact the spinal insert; and
removing the distractor.

26. The system of claim 25, wherein the spinal insert has a slot on either side to facilitate clasping by a distractor.

27. The system of claim 25, wherein the rod that is inserted into the spinal insert has a threaded distal end that is received by threading in a receiving hole on the spinal insert.

28. The system of claim 25, wherein the two blades on the distractor separate in parallel.

29. The system of claim 25, wherein the distractor is inserted between the vertebrae at an angle between 0 and 90 degrees.

30. The system of claim 25, wherein the distractor is inserted between the vertebrae at a 45 degree angle.

31. The system of claim 25, wherein the spinal insert has teeth on the portions of the spinal insert that contact the two vertebrae.

32. The system of claim 25, wherein the distractor has at least one hinge that allows for movement of an upper portion of the distractor.

33. The system of claim 25, wherein the spinal insert is substantially flat.

34. The system of claim 25, wherein the spinal insert is substantially arched in the center of the spinal insert.

35. The system of claim 25, wherein the spinal insert is substantially tapered from a proximal end to a distal end.
